(19) United States
(12) Patent Application Publication
(10) Pub. No.: US 2024/0417712 A1
(43) Pub. Date: Dec. 19, 2024

(54) SMALL DNA CLOCKS COMPRISING A COMPOSITION FOR MEASURING OR PREDICTING THE DURATION OF CELLULAR EVENTS IN CELLS

(71) Applicant: INDUSTRY-ACADEMIC COOPERATION FOUNDATION, YONSEI UNIVERSITY, Seoul (KR)

(72) Inventors: Hyongbum KIM, Seoul (KR); Ji-Hye PARK, Seoul (KR); Hanahrae LEE, Seoul (KR)

(21) Appl. No.: 18/741,521

(22) Filed: Jun. 12, 2024

(30) Foreign Application Priority Data
Jun. 13, 2023 (KR) 10-2023-0075530

(51) Int. Cl. C12N 9/78; C12N 9/22; C12N 15/11; C12Q 1/44

(52) U.S. Cl. CPC C12N 9/78; C12N 9/22; C12N 15/111; C12Q 1/44; C12Y 305/04004

(57) ABSTRACT

Please enter in the present application the English translation of the abstract of the Korean language application that was filed in the USPTO on Jun. 12, 2024, as contained in the English translation of the application in Appendix B of the Translator Certificate filed herewith, and as separately filed herewith in .docx format (728_Patent_Application_ABSTRACT.docx).

### CROSS-REFERENCE TO RELATED APPLICATION

The priority of Korean Patent Application 10-2023-0075530 filed Jun. 13, 2023 is hereby claimed under the provisions of 35 USC § 119, and the disclosure thereof is hereby incorporated herein by reference in its entirety, for all purposes.

### SEQUENCE LISTING

This application includes an electronically submitted sequence listing in .xml format. The .xml file contains a sequence listing entitled “728_CorrectedSeqListing.xml” created on Aug. 26, 2024 and is 4,727,392 bytes in size. The sequence listing contained in this .xml file is part of the specification and is hereby incorporated by reference herein in its entirety.

### BACKGROUND OF THE INVENTION

### Field of the Invention

The present invention relates to a small DNA clock comprising a composition for measuring or estimating elapsed time in cells, and a method of measuring or estimating elapsed time in cells using the composition.

### Description of the Related Art

Chromosomal DNA provides an excellent means for writing biological information as well as storing the same in appropriate “memory” devices.

This DNA is not only structurally durable, but also has advantages such as compatibility and cost-effectiveness. DNA is an excellent storage medium, and its storage capacity can be amplified by a variety of molecular biology tools. With advances in biological systems, it is extremely important to keep track of multiple simultaneously occurring biological activities. DNA writers are genetic devices that have dual functions: writing the code of life as well as enabling to bring about modifications in the living cells through mechanisms like base substitutions, deletions, inversions and insertions.

Biological life is one of the most complex and dynamic systems in nature. Through evolution and natural selection, vast biochemical and biological diversity has emerged, from complex molecules to multicellular life. These multi-scale biological systems precisely generate and respond to a myriad of biotic signals of varying order and magnitude. Signals can take the form of ions, metabolites, nucleic acids or proteins, producing biochemical gradients and signaling cascades that propagate across many length and time scales within cells and across populations. The integration of these signals through genetic and epigenetic regulation at the transcriptional, translational and post-translational levels results in robust cellular behaviors.

The diverse and enormous number of signals inducing changes in the cell is extremely difficult to be kept track of. With the advent of genomics, DNA can be used as an excellent writing means as well as a storage medium for overcoming the stereotypic difficulties associated with traditional methods of storing biological information (Science. 2018 Aug. 31; 361(6405): 870-875).

DNA is the fundamental molecule by which information is stored and utilized to produce life. DNA is a high-density storage medium that can be quickly copied by exponential polymerase chain reaction (PCR) amplification and stably preserved. Biological information encoded in DNA can be directly converted into actionable cellular responses through gene regulation and expression.

Many molecular events that occur in biological systems are transient and thus difficult to monitor and study within their native context. However, DNA writing can be used to create molecular recorders that capture these transient signals and stably encode them into the DNA of cell populations or individual cells. Although gene regulation and gene expression mechanisms can be utilized to convert biological activities into cellular signals, these signals have to be stored in a specific format. Nucleic acid sequencing and Next Generation Sequencing (NGS) have harnessed several components of cell lifecycle events like adaptive immunity, phase variation systems, arrangements in the genome, and retron-mediated recording systems. Thereamong, one of the most popular molecular recording devices is the CRISPR Cas-based molecular recording device.

Under this technical background, the present inventors have found that it is possible to advance an elapsed-time measurement system by increasing the number of targets in a single cell, thereby completing the present invention.

### SUMMARY OF THE INVENTION

An object of the present invention is to provide a composition for measuring or estimating elapsed time in isolated cells.

Another of the present invention is to provide a method for measuring or estimating elapsed time in cells using the composition.

To achieve the above objects, the present invention provides a composition for measuring or estimating elapsed time in isolated cells, comprising:
(a) a single-base editor or a nucleic acid encoding the same; and
(b) a guide RNA (gRNA) that targets human genome targets comprising, as a protospacer adjacent motif (PAM), NRG (where N is A, T, C, or G, and R is A or G) in cells, wherein the number of targets that are targeted by the gRNA is 10 to 10,000.

The present invention also provides isolated cells having introduced therein:
(a) a nucleic acid that encodes a single-base editor so that the single-base editor is expressed or is planned to be expressed; and
(b) a guide RNA (gRNA) that targets human genome targets comprising, as a protospacer adjacent motif (PAM), NRG (where N is A, T, C, or G, and R is A or G) in cells, wherein the number of targets that are targeted by the gRNA is 10 to 10,000.

The present invention also provides a method for measuring or estimating elapsed time in isolated cells, comprising steps of:
transducing the composition into isolated cells and then culturing the cells;
harvesting the cultured cells at any time point (t), and then measuring the copy number frequency of a sequence edited by the composition, that is, the A-to-G conversion frequency (CFᵢ) at i^{th} target site;
measuring the copy number frequency of an intact sequence in the total copy number of a target sequence, that is, the frequency of intact sequence (Fᵢ) at i^{th} target site; and
calculating the time elapsed from a given time point using the following equation:

$${F}_{i} = 1 - {CF}_{i} = {e}^{- {λ}_{i} ( t - {t}_{0} )} ( t \geq 0 , {t}_{0} \geq 0 )$$

wherein Fᵢ represents the frequency (fraction) of the copy number of an intact sequence at an i^{th} target site relative to the total copy number, analyzed at any time point, CFᵢ represents the copy number fraction of an edited sequence at the i^{th} target site, measured at any time point, λi is a positive constant that represents the rate of editing at the i^{th} target site per unit time, and t₀ is the latent time taken for the composition transduced into the cells to be expressed.

### BRIEF DESCRIPTION OF THE DRAWINGS

FIG. 1 is a graph showing the copy number of targets of guide RNAs (gRNAs) that target human repeat sequences.

FIG. 2 shows an overall pipeline for selecting TE-targeting guide RNAs.

FIG. 3 shows the results of selecting guide RNAs for small DNA clocks, which target retrotransposons that are expressed in lung adenocarcinoma cells lines and can be analyzed by single-cell RNA sequencing (scRNA-seq). FIGS. 3(A), 3(B) and 3(C) depict graphs showing the number of targets of each RNA guide and the sum of the expression levels of target sequences for each guide RNA in A549 (A), NCI-H1299 (B), and NCI-H1975 (C) cell lines (n=80,817). The Y-axis represents the sum of the expression levels of target gene sequence, obtained CCLE's bulk RNA sequencing data (RSEM data).

FIG. 4 shows the results of selecting guide RNAs with high expression levels in each section after dividing the guide RNAs into various sections based on the number of target copies in the human genome. In FIGS. 4(A), 4(B) and 4(C), among guide RNAs (n=3,716) with a wide range of target numbers in cell lines A549 (A), NCI-H1299 (B), and NCI-H1975 (C), selected guide RNAs that can be analyzed by scRNA-seq and have high RNA expression levels are indicated by blue or red dots (A549, n=43; NCI-H1299, n=52; NCI-H1975, n=49), guide RNAs contained only in individual cell lines are indicated by blue dots, and guide RNAs commonly contained in the three cell lines are indicated by red dots (n=37).

FIG. 5 shows the results of comparing public scRNA-seq data of TE-targeting guide RNAs in A549. (Left) a graph comparing the number of gene transcripts containing target sequences in A549 cell line, selected from CCLE data (x-axis), and the sum of transcript reads with target sequences detected from the results of public scRNA-seq of A549 cells, published in other papers (y-axis). A total of 9 guide RNAs with a high sum of the expression levels of transcripts containing target sequences were finally selected (indicated by red dots). (Right) a graph showing the number of genomic target targets for each guide RNA (x-axis), and the sum of the bulk RNA sequencing expression levels of target sequences for each guide RNA, extracted from the bulk RNA sequencing data of the CCLE database (y-axis).

FIG. 6 shows a construct for a “small DNA clock”.

FIG. 7 shows the results of a preliminary experiment conducted to analyze edits caused by SECURE-ABE-V82G and ABE9 variants in reporter target sequences of all selected TE-targeting guide RNAs A to J.

FIG. 8 shows the edit patterns of a region (target window), which can be targeted by the ABE9 variant, among reporter target sequences for guide RNAs H and I induced by ABE9.

FIG. 9 shows the results of testing each of guide RNAs A, F, I and J with the ABE9 variant and the dABE9 (dead ABE9) variant in human and mouse cells.

FIG. 10 shows the reporter target sequence editing results obtained by testing the selected guide RNA H and I with the ABE9 variant in A549 cells.

FIG. 11 shows the results of DNA and RNA editing analysis for three endogenous targets in A549 cells delivered with the selected guide RNA I and the ABE9 variant. Endogenous target sequences are denoted as endogenous target 1, 2, and 3. The two columns on the left show the results of analyzing the target sequence in DNA, and the two columns on the right show the results of editing analysis based on the RNA sequence. Two replicate experiments were performed.

FIG. 12 shows the results of estimating elapsed time using the selected guide RNA I. The left part of FIG. 12 shows the results of setting the mathematical function model for time to an exponential model, calculating the half-life of guide RNA I based on DNA editing changes, and estimating elapsed time by fitting the DNA editing changes, the center of FIG. 12 shows the results of estimating elapsed time by fitting RNA editing changes, and the right part of FIG. 12 shows the results of calculating the half-life of guide RNA I based on RNA editing changes and estimating elapsed time by fitting RNA editing changes.

### DETAILED DESCRIPTION OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

The strategy of targeting repeat elements is thought to be the optimal approach to accurately record time using only a small number of cells because it can simultaneously accumulate mutations in numerous target sequences in a single cell. Accordingly, to develop a “small DNA clock” system, the present inventors used a base editor (BE) system, which is convenient for gene editing. Unlike the Cas9 nuclease used in existing DNA clocks, which has the potential to cause double-strand breaks (DSBs) in DNA, causing extensive unwanted genomic modifications, BE protein can accurately induce mutations in the target sequence in the form of A-to-G or C-to-T conversion and work without causing DSBs. Types of BE proteins include an adenine base editor (ABE), which converts adenine (A) to guanine (G), and a cytosine base editor (CBE), which converts cytosine (C) to thymine (T). Thereamong, the adenine base editor (ABE), which converts adenine (A) to guanine (G), was used in a “small DNA clock”.

To construct the “small DNA clock”, repeat sequences present in the human genome were used as target sequences. To select guide RNAs targeting repeat sequences, 20-bp guide RNA sequences that include an NGG PAM were extracted from the Dfam database. The number of TE-targeting sequences selected from each database is 160,000. As a result of calculating the number of sequences in the human reference genome, which are targeted by the guide RNAs that recognize an NRG PAM (which is a PAM comprising NGG and NAG and it is known that guide RNAs can act not only on NGG but also on NAG PAM), the distribution of the guide RNAs is shown in FIG. 1.

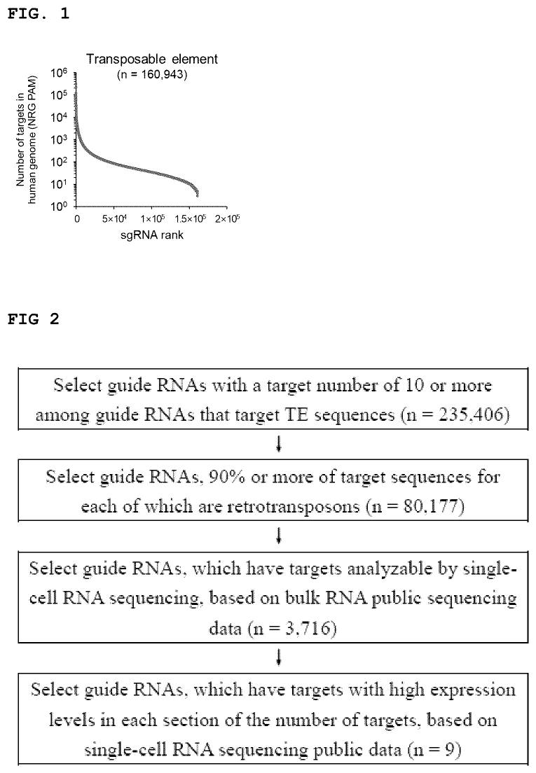

To select the optimal transposable element (TE)-targeting guide RNAs for use in the “small DNA clock”, the process of filtering guide RNAs was performed according to the pipeline shown in FIG. 2. The criteria for preferential selection of guide RNAs are as follows: 1) among the guide RNAs selected above, those whose target sequences are present in portions where they are transcribed into RNAs so that the target sequences can be detected by RNA sequencing; 2) guide RNAs whose target sequences have sufficiently high RNA expression levels in a specific type of cells and are detected with high efficiency by RNA sequencing; and 3) guide RNAs whose target sequences are abundant, which can be analyzed even by scRNA-seq in the future.

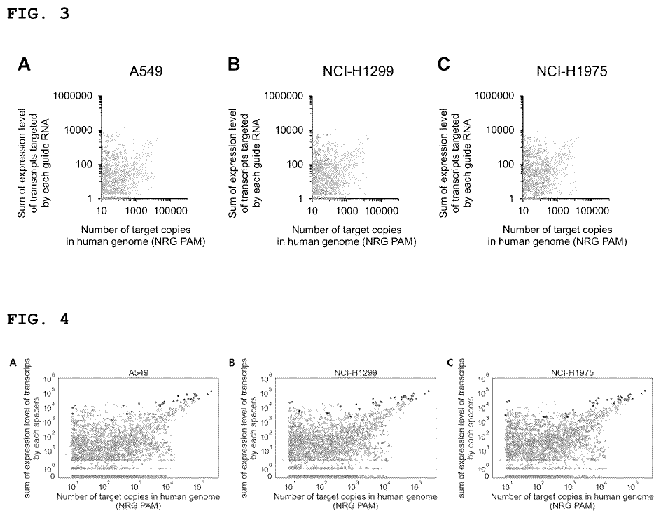

Among 160,000 guide RNAs that target TE, the present inventors selected about 80,000 guide RNA sequences whose target sequences belong to class I retrotransposons, can be targeted by an adenine base editor (ABE), and have adenine (A) without consecutive thymine (T) residues in the target window. In order to examine the RNA expression levels of target sequences for each guide RNA, location information of each target sequence on the human genome was examined, gene expression levels in A549, NCI-H1299 and NCI-H1975 cell lines were extracted using bulk RNA sequencing data retrieved from the CCLE (Cancer Cell Line Encyclopedia) database, and then the expression levels of genes corresponding to the target sequences for each guide RNA were summed. It was confirmed that, as the number of target sequences for each guide RNA increased, the sum of the expression levels of the target sequences tended to increase (FIG. 3).

To find guide RNAs that can detect target sequences in three cell lines by scRNA-seq, a total of 3,716 guide RNAs that target sequences within 200 bp from the 3′-end with respect to mature RNA sequences were selected based on the CCLE bulk RNA sequencing data (FIGS. 2 and 4). Among the selected guide RNAs that target various numbers of genomic targets, guide RNAs with high expression levels in each cell line were selected (FIG. 4, blue and red dots). Thereamong, a total of 37 guide RNAs commonly selected from the three cell lines were used for further analysis (FIG. 4, red dots).

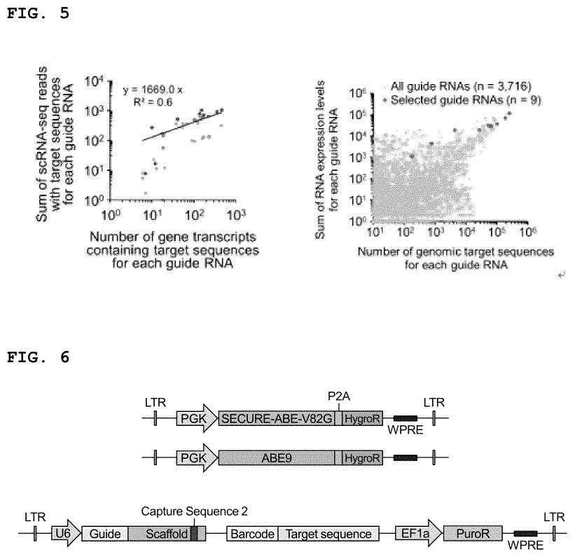

To examine whether the selected guide RNAs can detect target sequences when actually analyzed by scRNA-seq, additional analysis was performed in the A549 cell line using previously reported public scRNA-seq data (Wang C et al., American Society for Microbiology (2020)) (FIG. 2). The present inventors analyzed the number of reads detected by scRNA-seq in the target sequences for the 37 guide RNAs selected as described above (FIG. 5, left). It was confirmed that the number of genes containing target sequences for each guide RNA and detected by scRNA-seq (x-axis) was positively correlated with the number of scRNA-seq reads.

Thereamong, the present inventors selected 9 final guide RNAs that target a wide range number of genomic targets and show a large number of reads detected by scRNA-seq and high expression levels (FIG. 5, red dots).

Based on this, the present invention provides a composition for measuring or estimating elapsed time in cells, comprising:
(a) a single-base editor or a nucleic acid encoding the same; and
(b) a guide RNA (gRNA) that targets human genome targets comprising, as a protospacer adjacent motif (PAM), NRG (where N is A, T, C, or G, and R is A or G) in cells, wherein the number of targets that are targeted by the gRNA is 10 to 10,000.

The present invention also provides isolated cells having introduced therein:
(a) a nucleic acid that encodes a single-base editor so that the single-base editor is expressed or is planned to be expressed; and
(b) a guide RNA (gRNA) that targets human genome targets comprising, as a protospacer adjacent motif (PAM), NRG (where N is A, T, C, or G, and R is A or G) in cells, wherein the number of targets that are targeted by the gRNA is 10 to 10,000.

The present invention includes a single-base editor or a nucleic acid encoding the same.

The single-base editor comprises a polypeptide that is capable of making a modification to a base (e.g., A, T, C, G, or U) within a nucleic acid sequence (e.g., DNA or RNA). The single-base editor is capable of deaminating a base within a nucleic acid such as a base within a DNA molecule.

The single-base editor may be an adenine base editor comprising: a nickase or nuclease-inactivated dead editor for A-to-G editing; and an adenosine deaminase.

The adenine base editor can deaminate adenine (A) in DNA. The base editor may comprise a nucleic acid programmable DNA binding protein (napDNAbp) fused to an adenosine deaminase.

The single-base editor comprises a CRISPR-mediated fusion protein that is utilized in the base editing method. The single-base editor may comprise nuclease-inactive Cas9 fused to a deaminase that binds to a nucleic acid in a guide RNA-programmed manner.

The term “deaminase” or “deaminase domain” refers to a protein or enzyme that catalyzes a deamination reaction. The deaminase is an adenosine deaminase, which catalyzes the hydrolytic deamination of nucleobase adenine. The adenosine deaminase catalyzes the hydrolytic deamination of adenine in deoxyribonucleic acid (DNA) to hypoxanthine. The deaminases may be from any organism, such as a bacterium. In some embodiments, the deaminase or deaminase domain is a variant of a naturally-occurring deaminase from an organism. In some embodiments, the deaminase or deaminase domain may not occur in nature. For example, in some embodiments, the deaminase or deaminase domain may have a sequence identity of at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% to a naturally-occurring deaminase.

The adenosine deaminase may be derived from a bacterium, such as E. coli, S. aureus, S. typhi, S. putrefaciens, H. influenzae, or C. crescentus. In some embodiments, the adenosine deaminase is a TadA deaminase.

In a specific embodiment, the adenosine deaminase may comprise V82G mutation or the like in TadA-8e. For example, the adenosine deaminase may comprise the sequence of SEQ ID NO: 3717.

In addition, the adenosine deaminase may comprise an ABE9 variant, a dABE9 variant, etc. For example, the adenosine deaminase may comprise the sequence of SEQ ID NO: 3718.

The single-base editor may be a cytosine base editor comprising: a nickase or dead editor for C-to-T editing; and a cytidine deaminase.

The deaminase is a cytidine deaminase, which catalyzes the hydrolytic deamination of the nucleobase cytosine. Examples of the cytidine deaminase include apolipoprotein B editing complex 1 (APOBEC1) and activation-induced deaminase (AID). Most DNA deaminases may act only on single-stranded DNA and thus may not be suitable for base editing through linkage to a DNA-binding protein. Specifically, the cytidine deaminase may be derived from a deaminase (DddA) or an orthologue thereof that acts on double-stranded DNA. More specifically, the cytidine deaminase may be double-stranded DNA-specific bacterial cytidine deaminase.

The editor comprises nickase or a dead editor. The nickase may be a variant of Cas protein modified to nick a single strand of DNA, and the dead editor may be a variant of Cas protein modified to bind to a target sequence but not induce DNA nicking.

The Cas protein may be Cas1, Cas1B, Cas2, Cas3, Cas4, Cas5, Cas6, Cas7, Cas8, Cas9, Cas10, Cas12a, Cas12b, Cas12c, Cas12d, Cas12e, Cas12g, Cas12h, Cas12i, Cas12j, Cas13a, Cas13b, Cas13c, Cas13d, Cas14, Csy1, Csy2, Csy3, Cse1, Cse2, Csc1, Csc2, Csa5, Csn2, CsMT2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3 or Csf4 endonuclease, without being limited thereto.

The Cas protein may be derived or isolated from a Cas protein ortholog-containing microorganism selected from the group consisting of Corynebacter, Sutterella, Legionella, Treponema, Filifactor, Eubacterium, Streptococcus (Streptococcus pyogenes), Lactobacillus, Mycoplasma, Bacteroides, Flaviivola, Flavobacteriurn, Azospirillurn, Gluconacetobacter, Neisseria, Roseburia, Parvibaculurn, Staphylococcus (Staphylococcus aureus), Nitratifractor, Corynebacterium, and Campylobacter. Alternatively, the Cas protein may be a recombinant protein.

The Cas protein may be Streptococcus pyogenes Cas9 (SpCas9). Recognition of a protospacer adjacent motif (PAM) recognition by SpCas9 is the critical first step of target DNA recognition, enabling SpCas9 to bind to and hydrolyze DNA. SpCas9, the most robust and widely used Cas9, primarily includes NGG PAMs.

The variant of Cas protein is a variant retaining the function of Cas nuclease, and examples thereof include, but are not limited to, xCas9, SpCas9-NG, Cas9 nickase (nCas9), deactivated Cas9 (dCas9), and destabilized Cas9 (DD-Cas9).

The nickase may be one in which at least one amino acid selected from the group consisting of D10, E762, H839, H840, N854, N863, and D986 of Cas9 is substituted with a different amino acid.

Streptococcus pyogenes Cas9 may contain a mutation in which at least one selected from the group consisting of catalytic aspartate residue at position 10 (D10), glutamic acid at position 762 (E762), histidine at position 840 (H840), asparagine at position 854 (N854), asparagine at position 863 (N863), and aspartic acid at position 986 (D986) is substituted with any different amino acid. Here, any different amino acid for substitution may be alanine, without being limited thereto.

In some embodiments, the Streptococcus pyogenes Cas9 protein may be mutated to recognize NGA (where N is any base selected from among A, T, G, and C), which is different from the PAM sequence (NGG) recognized by wild-type Cas9, by substituting at least one selected from among aspartic acid at position 1135 (D1135), arginine at position 1335 (R1335), and threonine at position 1337 (T1337), for example, all of the three amino acids, with a different amino acid.

For example, in the amino acid sequence of the Streptococcus pyogenes Cas9 protein, amino acid substitution may occur at
(1) D10, H840, or D10+H840;
(2) D1135, R1335, T1337, or D1135+R1335+T1337; or
(3) both residues (1) and (2).

The term “different amino acid” means an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all known variants of the amino acids thereof, exclusive of the amino acid found at the original mutation positions in the wild-type protein. In one example, the “different amino acid” may be alanine, valine, glutamine, or arginine.

In a specific embodiment according to the present invention, the nickase may be nSpCas9 (D10A) or dSpCas9 (D10A/H840A). The nickase may be nSpCas9 (D10A) comprising the sequence of SEQ ID NO: 3719 or dSpCas9 (D10A/H840A) comprising the sequence of SEQ ID NO: 3720.

The present invention may further include a guide RNA. The guide RNA may be, for example, at least one selected from the group consisting of CRISPR RNA (crRNA), trans-activating crRNA (tracrRNA), and single guide RNA (sgRNA). Specifically, the guide RNA may be a double-stranded crRNA:tracrRNA complex in which crRNA and tracrRNA are linked to each other, or a single-stranded guide RNA (sgRNA) in which crRNA or a portion thereof and tracrRNA or a portion thereof are linked together by an oligonucleotide linker.

Herein, “editing” may be used interchangeably with “edit” or “edited”, and refers to a method of altering the nucleic acid sequence of a specific genomic target. Such a specific genomic target includes, but is not limited to, a chromosomal region, a gene, a promoter, an open reading frame, or any nucleic acid sequence.

The number of targets that are targeted by the gRNA may be 10 to 10,000, specifically 100 to 2,000, more specifically 300 to 1,000.

The gRNA comprises at least one sequence selected from the group consisting of SEQ ID NOs: 1 to 3716.

The “protospacer adjacent motif (PAM)” is a sequence essentially required for the Cas protein to bind to target DNA, and refers to the sequence located after the target nucleic acid. Bacteria store a portion of the sequence of an invading virus in a part of their genome, and this sequence is called the protospacer. Since the protospacer sequence is a partial sequence, the sequence adjacent thereto is the original sequence of the bacteria. The role of the PAM site is to prevent a sequence from being cleaved, unless the sequence is not flanked by the PAM, even though many sequences in bacteria may match the protospacer.

The elapsed time may be measured or estimated by a method comprising steps of:
(a) transducing the composition into isolated cells and then culturing the cells;
(b) measuring the copy number frequency of a sequence edited by the composition, that is, the A-to-G conversion frequency (CFᵢ) at an i^{th} target site;
(c) measuring the copy number frequency of an intact sequence in the total copy number of the edited sequence, that is, the frequency of intact sequence (Fᵢ) at the i^{th} target site; and
(d) measuring the time elapsed from a given time point using the following equation:

$${F}_{i} = 1 - {CF}_{i} = {e}^{- {λ}_{i} ( t - {t}_{0} )} ( t \geq 0 , {t}_{0} \geq 0 )$$

wherein Fᵢ represents the frequency (fraction) of the copy number of an intact sequence at the i^{th} target site relative to the total copy number, analyzed at any time point, CFᵢ represents the copy number fraction of the edited sequence at the i^{th} target site, measured at any time point, λi is a positive constant that represents the rate of editing at the i^{th} target site per unit time, and to is the latent time taken for the composition transduced into the cells to be expressed.

The method may further comprise a step of calculating λi using the following equation:

$${F}_{i} = {e}^{- {λ}_{i} {t}^{*}} ( {t}^{*} \geq 0 )$$

wherein Fᵢ represents the frequency (fraction) of the copy number of an intact sequence at the i^{th} target site relative to the total copy number, analyzed at any time point, λi is a positive constant, and t* is a positive constant that represents a given time point.

In the present invention, the step of measuring the copy number frequency of the edited sequence, that is, A-to-G conversion frequency (CFᵢ) at the i^{th} target site, comprises obtaining a DNA sequence from cells exhibiting the nuclease activity of the transduced composition. This step of obtaining the DNA sequence may be performed using various DNA isolation methods known in the art.

Since it is considered that editing in the target sequence has occurred in each of the transduced cells, data may be obtained by performing sequencing of the target sequence, for example, deep sequencing or RNA sequencing.

In the present invention, step (c) comprises measuring the copy number frequency of an intact sequence in the total copy number of the edited sequence, that is, frequency of intact sequence (Fᵢ) at i^{th} target site.

The following equation should be satisfied:

$${F}_{i} = 1 - {CF}_{i}$$

wherein Fi represents the frequency (fraction) of the copy number of an intact sequence at the i^{th} target site relative to the total copy number, analyzed at any time point, and CFi represents the copy number fraction of the edited sequence at the i^{th} target site, measured at any time point.

Then, in step (d), the time elapsed from a given time point in the cells is calculated using the following equation:

$${F}_{i} = 1 - {CF}_{i} = {e}^{- {λ}_{i} ( t - {t}_{0} )} ( t \geq 0 , {t}_{0} \geq 0 )$$

This time measurement method is based on the fact that the frequency of intact target sequences decreases exponentially over time. NGS analysis was performed to examine whether intended sequence editing at each target site occurred. The copy number frequency of an intact sequence relative to the total copy number at each target site over time, that is, the frequency of intact sequence (Fi) at the i^{th} target site, was calculated and fitted with an exponential decay curve.

To record elapsed time in isolated cells, an editing sequence is utilized. If the concentrations of the single-base editor protein and the gRNA are kept constant, the editing rate of the target sequence per unit time in individual cells is assumed to be constant and is expressed as lambda (λ).

When one target sequence is introduced per cell, reactions occur individually in each cell, and each edit in the target sequence is an independent event.

In this case, the rate of sequence editing, or the rate of decrease in the copy number of the intact target sequence in the entire cell population (λ), is linearly proportional to the copy number of the intact target sequence at time t (Nₜ), and may be expressed by the following equation:

$$\frac{{dN}_{t}}{dt} = - λ {N}_{t} ( λ > 0 ) \qquad equation ( 1 )$$

The definite integral equation for time t in equation (1) above is as follows:

$${F}_{t} = {e}^{- λt} \qquad equation ( 2 )$$

Here, Fₜ=Nₜ/Nₜ represents the fraction or relative frequency of the copy number of an intact target sequence in the total copy number of the target sequence at time t (hereinafter referred to as frequency), and Nₑ represents the initial (at time 0) copy number of the intact target sequence. As shown in equation (2) above, F_{i,t} follows the exponential decay that is used in radiometric dating.

The probability of decrease in the copy number of the intact target sequence per unit cell (λ) is determined by the sequence composition of the target sequence when introducing the target sequence using lentiviral transduction, and the concentrations of the single-base editor and the gRNA. Therefore, if the expression levels of the single-base editor and the gRNA are kept constant, λ is determined by the composition of the target sequence.

As used herein, the term “target” or “target site” refers to a pre-identified nucleic acid sequence of any composition and/or length. Such a target includes, but is not limited to, a chromosomal region, a gene, a promoter, an open reading frame, or any nucleic acid sequence.

As used herein, “on-target” refers to a subsequence of a specific genomic target that may be completely complementary to a programmable DNA binding domain and/or a guide RNA sequence.

The terms “polypeptide”, “peptide” and “protein” are used interchangeably to refer to a polymer of amino acid residues, wherein the polymer may be conjugated to a moiety that does not consist of amino acids in the examples. The terms may apply to amino acid polymers in which one or more amino acid residues are artificial chemical mimetics of corresponding naturally occurring amino acids, as well as to naturally occurring amino acid polymers and non-naturally occurring amino acid polymers. A “fusion protein” may refer to a chimeric protein encoding two or more separate protein sequences that are recombinantly expressed as a single moiety.

The term “amino acid” may refer to naturally occurring and synthetic amino acids, as well as amino acid analogs and amino acid mimetics that function in a manner similar to the naturally occurring amino acids. Naturally occurring amino acids include those encoded by the genetic code, as well as those amino acids that are later modified, e.g., hydroxyproline, γ-carboxyglutamate, and O-phosphoserine. Amino acid analogs include compounds that have the same basic chemical structure as a naturally occurring amino acid, i.e., a carbon that is bonded to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but may retain the same basic chemical structure as a naturally occurring amino acid. Amino acid mimetics include chemical compounds that have a structure that is different from the general chemical structure of an amino acid, but that function in a manner similar to a naturally occurring amino acid. The terms “non-naturally occurring amino acids” and “non-natural amino acids” include amino acid analogs, synthetic amino acids and amino acid mimetics, which are not found in nature.

The invention is also directed to a nucleic acid encoding the composition.

The term “nucleic acid” is used interchangeably with “oligonucleotides”, “polynucleotides”, “nucleotides” and “nucleotide sequences”. The term may include a polymeric form of nucleotides of any length, either deoxyribonucleotides or ribonucleotides, or analogs thereof. Polynucleotides may have any three-dimensional structure, and may perform any function, known or unknown. A polynucleotide may comprise modified nucleotides, such as methylated nucleotides and nucleotide analogs. If present, modifications to the nucleotide structure may be imparted before or after assembly of the polymer.

The polynucleotide may typically comprise four nucleotide bases: adenine (A), cytosine (C), guanine (G), and thymine (T) (uracil (U) for thymine (T) when the polynucleotide is RNA).

The term “sequence” is the alphabetical representation of a molecule. This alphabetical representation may be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “nucleic acid” may include deoxyribonucleotides or ribonucleotides and polymers in either single-, double- or multiple stranded form, or complements thereof. The term “polynucleotide” may include a linear sequence of nucleotides.

The nucleic acid may be linear or branched. For example, the nucleic acid may be a linear chain of nucleotides, or the nucleic acid may be branched so that it makes up one or more nucleotide arms or branches.

The nucleic acid may be an RNA sequence, a DNA sequence, or a combination thereof (RNA-DNA combination sequence).

“Conservatively modified variations” may be applied to nucleic acid sequences. With respect to particular nucleic acid sequences, “conservatively modified variations” refers to those nucleic acids which encode identical or essentially identical amino acid sequences. Due to degeneracy of the genetic code, a large number of nucleic acid sequences can encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservatively modified variations. Every nucleic acid sequence which encodes a polypeptide may also include every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule.

The term “isolated”, when applied to a nucleic acid or protein, denotes that the nucleic acid or protein is essentially free of other cellular components. Purity and homogeneity may be typically determined using analytical chemistry techniques such as polyacrylamide gel electrophoresis or high-performance liquid chromatography.

“Complementarity” or “complementary” refers to the ability of a nucleic acid to form hydrogen bond(s) with another nucleic acid sequence by either traditional Watson-Crick base pairing or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, and 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary”, as used herein, refers to a degree of complementarity that is at least 60%, 65%, 70%, 75%, 80%, 85%, 90%, or 95%. “Perfectly complementary” refers to a degree of complementarity that is at least 90%, 91%, 92%, 93%, 94%, 95%, 96%, 97%, 98%, 99% or 100% over a region of nucleotides, or refers to two nucleic acids that hybridize under stringent conditions.

The term “gene” means the segment of DNA involved in producing a protein and may include regions preceding and following the coding region (leader and trailer) as well as intervening sequences (introns) between individual coding segments (exons). The leader, the trailer and the introns include regulatory elements that are necessary during transcription and translation of a gene. Further, a “protein gene product” includes a protein expressed from a particular gene.

The nucleic acid may be delivered using a viral vector, for example, an adeno-associated viral vector (AAV), an adenoviral vector (AdV), lentiviral vector (LV) or a retroviral vector (RV), as well as other viral vectors such as episomal vectors containing Simian virus 40 (SV40) ori, bovine papilloma virus (BPV) ori, or Epstein-Barr nuclear antigen (EBV) ori, as well as virus-like particles (VLPs) or engineered virus-like particles (eVLPs).

The vector may be delivered in vivo or into cells through microinjection (e.g., direct injection into a lesion or target site), electroporation, lipofection, viral vector, nanoparticles, protein translocation domain (PTD) fusion proteins, etc.

For delivery, a known expression vector such as a plasmid vector, a cosmid vector, or a bacteriophage vector may be used, and the vector may be easily produced by those skilled in the art according to any known method using DNA recombination technology. The vector may be a viral vector or a plasmid vector, and the viral vector may specifically be a lentiviral vector or a retroviral vector. However, the present invention is not limited thereto, and those skilled in the art can freely use known vectors as long as the purpose of the present invention can be achieved.

For viral vectors, virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell.

In certain cases, vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Moreover, certain vectors are capable of directing the expression of genes to which they are operatively linked. Such vectors are referred to herein as “expression vectors”. Common expression vectors of utility in recombinant DNA techniques are often in the form of plasmids.

Recombinant expression vectors may comprise a nucleic acid in a form suitable for expression of the nucleic acid in a host cell, which means that the recombinant expression vectors comprise one or more regulatory elements, which may be selected on the basis of the host cells to be used for expression, that is operatively-linked to the nucleic acid sequence to be expressed.

Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” may include promoters, enhancers, internal ribosomal entry sites (IRES), and other expression regulatory elements (e.g., transcription termination signals, such as polyadenylation signals and poly-U sequences). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific.

In some embodiments, a vector may comprise one or more pol III promoters, one or more pol II promoters, one or more pol I promoters, or combinations thereof. Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter.

The term “regulatory elements” may include enhancer elements, such as WPRE; CMV enhancers; and the intron sequence between exons 2 and 3 of rabbit β-globin. It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including encoded by nucleic acids as described herein (e.g., clustered regularly interspaced short palindromic repeat (CRISPR) transcripts, proteins, enzymes, mutants thereof, fusion proteins thereof, etc.). Advantageous vectors include lentiviruses and adeno-associated viruses, and types of such vectors can also be selected for targeting particular types of cells.

Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., β-galactosidase, alkaline phosphatase or luciferase), and genes which visibly affect the phenotype of transformed or transfected cells, hosts, colonies, or plaques. Any vector suitable for the transformation of a host cell, (e.g., E. coli, mammalian cells such as CHO cell, insect cells, etc.) as embraced by the present invention, for example vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing gRNAs and/or proteins (e.g., those provided herein), transforming cells, and expressing/purifying recombinant proteins are well known in the art.

Examples of the cells include, but are not limited to, eukaryotic cells (e.g., embryonic cells, stem cells, somatic cells, germ cells, etc.) derived from fungi such as yeast, eukaryotic animals, and/or eukaryotic plants, cells derived from eukaryotic animals (e.g., primates such as humans, monkeys, dogs, pigs, cattle, sheep, goats, mice, rats, etc.), or cells derived from eukaryotic plants (e.g. algae such as green algae, corn, soybeans, wheat, rice, etc.).

The DNA sequence encoding the single-base editor and the DNA sequence encoding the guide RNA may be provided through a delivery means such as a vector. The DNA sequence encoding the single-base editor and the DNA sequence encoding the guide RNA may be placed on the same vector, so that they may be delivered simultaneously by the single vector. The DNA sequence encoding the single-base editor and the DNA sequence encoding the guide RNA may be placed on different vectors and delivered by the vectors.

In some embodiments, the sequence encoding the single-base editor, and the guide RNA may be delivered in mRNA form. The mRNA may be delivered directly into cells or delivered by a carrier.

Furthermore, an RNP (ribonucleoprotein) complex formed by assembling the sequence encoding the single-base editor and the mRNA of the guide RNA may be delivered. The RNP may be delivered directly or delivered by a carrier.

The RNP complex may be delivered into cells by various methods known in the art, such as microinjection, electroporation, DEAE-dextran treatment, lipofection, nanoparticle-mediated transfection, protein transduction domain-mediated introduction, and PEG-mediated transfection, without being limited thereto.

The carrier may comprise, for example, a cell-penetrating peptide (CPP), nanoparticles, or a polymer, without being limited thereto. CPPs are short peptides that facilitate cellular uptake of a variety of molecular cargoes (from nanosized particles to small chemical molecules and large fragments of DNA). With respect to the nanoparticles, the composition according to the present invention may be delivered by polymer nanoparticles, metal nanoparticles, metal/inorganic nanoparticles, or lipid nanoparticles.

### EXAMPLES

Hereinafter, the present invention will be described in more detail with reference to examples. These examples are only for illustrating the present invention, and it will be apparent to those of ordinary skill in the art that the scope of the present invention is not to be construed as being limited by these examples.

### Example 1. Construction of “Small DNA Clock” System

To construct a “small DNA clock”, repeat sequences present in the human genome were used as target sequences. To select guide RNAs targeting repeat sequences, 20-bp guide RNA sequences that include an NGG PAM were extracted from the Dfam database. The number of TE-targeting sequences selected from each database was 160,000. As a result of calculating the number of sequences in the human reference genome, which are targeted by the guide RNAs that recognize an NRG PAM (which is a PAM comprising NGG and NAG and it is known that guide RNAs can act not only on NGG but also on NAG PAM), the distribution of the guide RNAs is shown in FIG. 1.

To select the optimal transposable element (TE)-targeting guide RNAs for use in the “small DNA clock”, the process of filtering guide RNAs was performed according to the pipeline shown in FIG. 2. The criteria for preferential selection of guide RNAs are as follows: 1) among the guide RNAs selected above, those whose target sequences are present in portions where they are transcribed into RNAs so that the target sequences can be detected by RNA sequencing; 2) guide RNAs whose target sequences have sufficiently high RNA expression levels in a specific type of cells and are detected with high efficiency by RNA sequencing; and 3) guide RNAs whose target sequences are abundant, which can be analyzed even by scRNA-seq in the future.

Among 160,000 guide RNAs that target TE, the present inventors selected about 80,000 guide RNA sequences whose target sequences belong to class I retrotransposons, can be targeted by an adenine base editor, and have adenine (A) without consecutive thymine (T) residues in the target window. In order to examine the RNA expression levels of target sequences for each guide RNA, location information of each target sequence on the human genome was examined, gene expression levels in A549, NCI-H1299 and NCI-H1975 cell lines were extracted using bulk RNA sequencing data retrieved from the CCLE (Cancer Cell Line Encyclopedia) database, and then the expression levels of genes corresponding to the target sequences for each guide RNA were summed. It was confirmed that, as the number of target sequences for each guide RNA increased, the sum of the expression levels of the target sequences tended to increase (FIG. 3).

To find guide RNAs that can detect target sequences in three cell lines by scRNA-seq, a total of 3,716 guide RNAs that target sequences within 200 bp from the 3′-end with respect to mature RNA sequences were selected based on the CCLE bulk RNA sequencing data (FIGS. 2 and 4). Among the selected guide RNAs that target various numbers of genomic targets, guide RNAs with high expression levels in each cell line were selected (FIG. 4, blue and red dots). Thereamong, a total of 37 guide RNAs commonly selected from the three cell lines were used for further analysis (FIG. 4, red dots).

To examine whether the selected guide RNAs can detect target sequences when actually analyzed by scRNA-seq, additional analysis was performed in the A549 cell line using previously reported public scRNA-seq data (Wang C et al., American Society for Microbiology (2020)) (FIG. 2). The present inventors analyzed the number of reads detected by scRNA-seq in the target sequences for the 37 guide RNAs selected as described above (FIG. 5, left). It was confirmed that the number of genes containing target sequences for each guide RNA and detected by scRNA-seq (x-axis) was positively correlated with the number of scRNA-seq reads.

Thereamong, the present inventors selected 9 final guide RNAs that target a wide range number of genomic targets and show a large number of reads detected by scRNA-seq and high expression levels (FIG. 5, red dots).

Because there is a report that if there is an excessively large number of targets on the genome, targeting by ABE or CBE can cause cell death, the present inventors sought to test guide RNAs that target various numbers of targets (Smith C J et al., Nucleic Acids Research (2020)). For the number of the final 9 TE-targeting guide RNAs (guide RNAs A to I), there are 604 to 300,000 targets in the human genome and 176 to 250,000 transposable element (TE) repeat sequences as targets in the human genome. In addition, a non-targeting guide RNA (guide RNA J) was included as a negative control. The present inventors also investigated the DeepABE score, which is a score indicating the editing efficiency predicted by deep learning.

TABLE 1

| | Guide | Number of Human | DeepABE | |
|---|---|---|---|---|
| ID | RNA sequence (20 bp) | genome targets | score | Note |
| A | TGTAATCCCAGCACTTTGGG | 301248 | 77.5 | |
| B | GCCTGTAATCCCAGCACTTT | 224377 | 49.5 | |
| C | CCCAGCACTTTGGGAGGCCG | 118573 | 31.9 | |
| D | CTAAAAATACAAAAATTAGC | 134299 | 0.9 | |
| E | TAAAAATACAAAAATTAGCC | 77954 | 9.9 | |
| F | TGAGACCAGCCTGGCCAACA | 37800 | 90.7 | |
| G | ATACAAAAATTAGCCGGGTG | 7474 | 13.1 | |
| H | GACTCAGCCCGCCTGCACCC | 967 | 37.9 | |
| I | GCTGTACAGGAAGCATGGCT | 604 | 81.9 | |
| J | CAATATCGGGTGCTACAGGA | 0 | 82.3 | non-targeting control |

### Example 2. Construct for “Small DNA Clock”

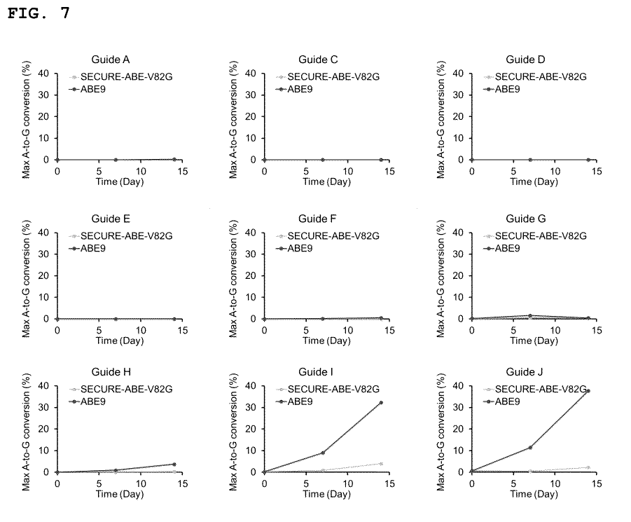

The present inventors designed a construct to experiment with observing A-to-G edits by delivering, into the A549 cell line, the nine selected guide RNAs A to J and two gene editors (SECURE-ABE-V82G variant (Grünewald J et al., Nature Biotechnology (2020)), and ABE9 variant (Chen L et al., Nature Chemical Biology (2023)) known to have few RNA off-targets (FIG. 6, upper panel). The sequence for SECURE-ABE-V82G having the construct NLS-TadA7.10-32AA linker-nSpCas9(D10A)-NLS comprises the sequence of SEQ ID NO: 3721. The sequence for the construct NLS-TadA8e-32AA linker-nSpCas9(D10A)-NLS comprises the sequence of SEQ ID NO: 3722. The sequence for the construct NLS-TadA8e-32AA linker-dSpCas9(D10A/H840A)-NLS comprises the sequence of SEQ ID NO: 3723.

To request scRNA-seq analysis from 10× Genomics in the future, a capture sequence was inserted into the scaffold sequence of the guide RNA. When performing scRNA-seq using the resulting construct, transcriptome data can be obtained from a single cell, and at the same time, the sequence of the guide RNA can be determined, and thus even if each cell is pooled and used, the type of guide RNA can be determined (FIG. 6, lower panel).

In order to easily determine whether editing occurred, the reporter target sequence was included in the same vector as the guide RNA, and future editing that occurred in the reporter target sequence was first analyzed.

### Example 3. Selection of Guide RNAs

For the selected guide RNAs, a preliminary experiment was performed in the A549 cell line to first check which sequence produces significant editing with less cytotoxicity and to select the best guide RNAs among the guide RNAs. Guide RNA vectors were constructed with lentivirus, and each of the vectors was transduced into each A549 cell line and integrated into the genome, followed by selection with puromycin. To determine how much A-to-G editing occurred in the reporter target sequence, the samples were analyzed 7 and 14 days after hygromycin B selection after ABE delivery. Guide RNA B was not cloned well, and thus was excluded from the experiment.

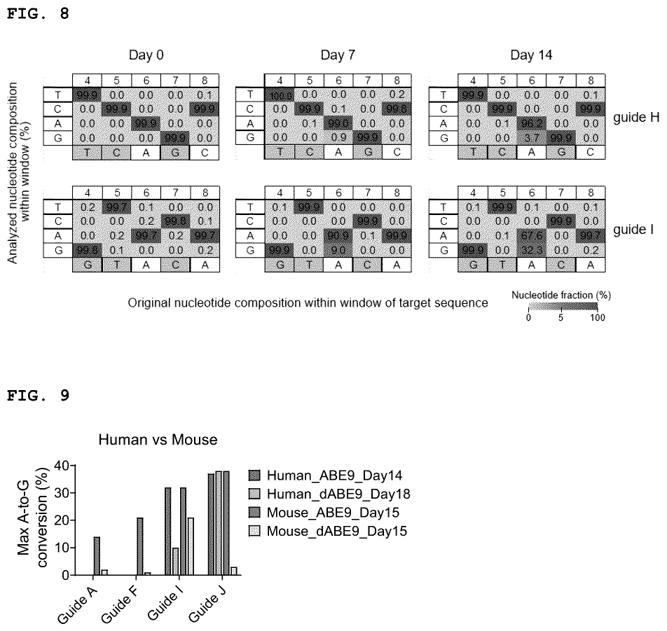

Editing was rarely observed in guide RNAs A to G, which had a relatively large number of targets (4,000 or more targets), and editing tended to increase in guide RNAs that had a relatively small number of targets (less than 800 targets). Among the two ABE variants tested, the ABE9 variant exhibited higher efficiency than the SECURE-ABE-V82G variant (FIG. 7). Based on the edit patterns generated in the reporter target sequences of guide RNA H and I using the ABE9 variant, it was observed that the A-to-G editing efficiency on day 14 in A present within the target window was 3.7% in guide RNA H and was as high as 32.3% in guide RNA I (FIG. 8).

An experiment was conducted to determine whether the reason why no significant editing was observed in guide RNAs A to F is because the activity of the guide RNAs was low in the first place, or whether the reason is because cells in which editing occurred underwent cell death due to genotoxicity caused by an excessively large number of targets on the genome as previously reported (Smith C J et al., Nucleic Acids Research (2020)). In addition, according to the same paper, there is a report that if a dead ABE variant that has lost the nickase function is used, higher editing efficiency can be achieved by reducing genotoxicity using a guide RNA having many targets.

As a result of analyzing target sequences for each guide RNA in the mouse cell genome, it was confirmed that there were almost no endogenous targets in the mouse cell line, unlike the human genome (Table 2). Therefore, for guide RNAs A, F, I and J with different target numbers among guide RNAs with high DeepABE scores, a validation experiment was performed using ABE9 and dead ABE9 (dABE9).

TABLE 2

| | human gene | mouse gene |
|---|---|---|
| Guide | target number | target numer |
| A | 301,248 | 2 |
| C | 118,573 | 4 |
| D | 134,299 | 0 |
| E | 77,954 | 0 |
| F | 37,800 | 0 |
| G | 7,474 | 0 |
| H | 967 | 0 |
| I | 604 | 0 |

The results of inducing editing by the ABE9 variant and the dABE9 variant in each of the human cell line A549 and the mouse cell line N2A for the same guide RNAs A, F, I and J are as follows (FIG. 9). First, it was observed that the editing efficiency of the dABE9 variant in both the human and mouse cells was significantly lower than that of the ABE9 variant, or that the dABE9 variant produced almost no edits in the mouse cells, except for guide RNA I. As expected, editing was observed in all of the guide RNAs in the mouse cells without endogenous targets for each guide RNA. Therefore, taking these results together, it can be determined that an actual editing event occurred even in the guide RNA that had a large number of targets in the A549 cells, but there seemed to be little editing efficiency due to genotoxicity.

Therefore, as a further experiment, the present inventors conducted an experiment to observe editing at narrower intervals of time points using guide RNAs G to I, which have a relatively small number of targets, and the ABE9 variant. Since guide RNAs G to I also have 604 to 7,474 targets, it is believed that guide RNAs G to I have a sufficient number of targets to construct a “small DNA clock”.

### Example 4. Confirmation of Editing in Human Genome

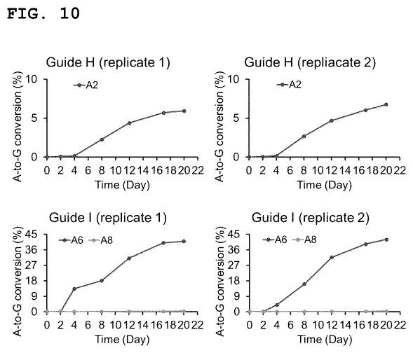

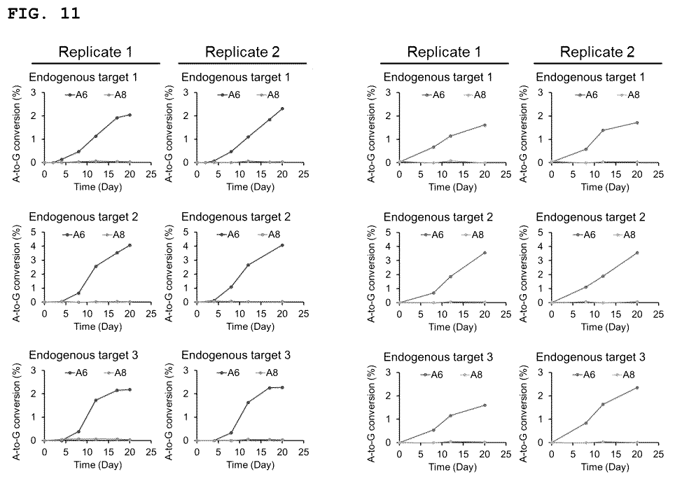

An experiment was conducted to check whether similar edits were observed not only in the reporter target sequence but also in the endogenous repeat target sequence on the actual human genome. Guide RNA G, H and I, selected based on the above-described results, and the ABE9 variant were transduced into A549 cells by lentivirus, the number of initial time points was increased, and edits that occurred in the reporter target sequence were closely analyzed. As a result, highly reproducible results similar to the results of the preliminary experiment could be confirmed (FIG. 10). To analyze edits that occurred in the endogenous genomic target sequence in the same sample, the edits were analyzed by NGS using PCR primers that amplify each of three targets with the highest expression levels for each guide RNA.

As a result of analyzing three endogenous target sequences in each of DNA and RNA for the guide RNA I sample, an increase in A-to-G editing in all endogenous target sequences could be observed in both DNA and RNA (FIG. 11). It was confirmed that the rate of editing observed in RNA targets was consistently slightly higher than that in DNA targets. A similar tendency was also found in other papers that analyzed the editing rate in both DNA and RNA (Smith C J et al., Nucleic Acids Research (2020)).

In the case of guide RNA H, the reporter target editing efficiency was lower due to a larger number of target sequences for guide RNA H, and thus it could be confirmed that the editing rate on day 8, a relatively early time point, was still very low (editing efficiency of up to about 0.3% in both endogenous target sequence DNA and RNA). However, since editing tended to increase in DNA and RNA in endogenous targets, it is expected that a higher degree of editing will be observed over time.

### Example 5. Checking of Elapsed Time

The results of Example 4 suggest that, in addition to editing occurring in the inserted reporter target sequence, editing also occurs cumulatively in numerous other endogenous targets present within the cell.

Although the editing efficiency was lower than that in the reporter target sequence, both DNA and RNA editing tended to gradually increase over time. The present inventors are currently analyzing a mathematical model based on these data. It is expected that the construct of the present invention will work not only as a “small DNA clock” but also as a “small RNA clock”. This suggests that, if the same sample is analyzed for editing in more target sequences at longer time points in the future, there is a clear possibility that accurate time estimations can be made using a small number of cells.

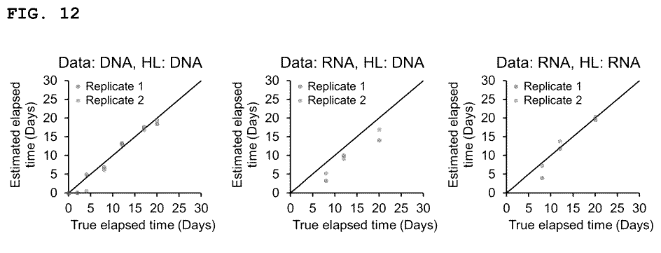

As a result of estimating elapsed time based on the exponential decay mathematical model for the A-to-G edit information generated in each endogenous target for guide RNA I, it was found that elapsed time was estimated well up to a period of about 20 days (FIG. 12).

As a result of either calculating the half-life based on the result of DNA editing and then estimating elapsed time using DNA (FIG. 12, left), or calculating the half-life based on the result of RNA editing and then estimating elapsed time using RNA (FIG. 12, right), it was found that the exponential decay model appeared to be suitable for use in time estimation, and the accuracy of time estimation values was high. In the case of the graph (FIG. 12, center) obtained by calculating the half-life based on the result of DNA editing and then estimating elapsed time using RNA, the estimated value appears to be slightly lower due to the difference in RNA expression level, but it is believed that the time estimation value can be corrected to a more accurate value through additional experiments.

As a result, the small DNA clock may use a base editor (ABE, CBE, etc.) to target the sequences present in the nucleic acid of human cells, and up to 350,000 repeat elements within the cell may be used as target sequences. Although the experiment was conducted using ABE, CBE with a similar mechanism can be used if cytosine exists within the window of the target sequence (Komor A C et al., Nature (2016), Gaudelli N M et al., Nature (2017)).

Since single cell RNA sequencing (scRNA-seq) technology should be applied so that editing changes in many target sequences can be simultaneously detected in a single cell, sequences present within 200 bp from the 3′-end of the RNA transcripts were selected as target sequences. As a result of filtering under the corresponding conditions, 3,716 guide RNAs that can read the target sequences through scRNA-seq were selected (FIG. 2).

Among the selected guide RNAs, 9 guide RNAs with high RNA expression levels were finally selected by dividing the guide RNAs into various sections according to the number of genomic targets. As a result of conducting the preliminary experiment, editing was not observed for guide RNAs with 30,000 or more targets (FIG. 7).

It can be considered that the reason why editing was not observed even after sufficient time had passed in guide RNAs with many targets was not because the efficiency of the guide RNAs was low, but because of genotoxicity caused by a large number of targets, even though an actual editing event occurred (FIG. 9).

Therefore, it was determined that when a guide RNA with a large number of targets in a single cell was used in the “small DNA clock”, the number of targets where minimal editing can be observed was about 10,000 or less (corresponding to guide RNA G). When the number of targets is less than about 1,000, it is easier to measure edits over time (corresponding to guide RNA H and I).

When the number of genome targets is very small (less than 10), the number of targets that can be detected by scRNA-seq is less than 10, and thus it is difficult to accurately estimate elapsed time based on editing information of the targets.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

What is claimed is:

1. A composition for measuring or estimating elapsed time in isolated cells, comprising:
(a) a single-base editor or a nucleic acid encoding the same; and
(b) a guide RNA (gRNA) that targets human genome targets comprising, as a protospacer adjacent motif (PAM), NRG (where N is A, T, C, or G, and R is A or G) in cells, wherein the number of targets that are targeted by the gRNA is 10 to 10,000.

2. The composition according to claim 1, wherein the gRNA targets transposable elements (TEs).

3. The composition according to claim 1, wherein the single-base editor is an adenine base editor comprising: a nickase or dead editor for A-to-G editing; and an adenosine deaminase.

4. The composition according to claim 1, wherein the single-base editor is a cytosine base editor comprising: a nickase or dead editor for C-to-T editing; and a cytidine deaminase.

5. The composition according to claim 1, wherein the gRNA comprises at least one sequence selected from the group consisting of SEQ ID NOs: 1 to 3716.

6. Isolated cells having introduced therein:
(a) a nucleic acid that encodes a single-base editor so that the single-base editor is expressed or is planned to be expressed; and
(b) a guide RNA (gRNA) that targets human genome targets comprising, as a protospacer adjacent motif (PAM), NRG (where N is A, T, C, or G, and R is A or G) in cells, wherein the number of targets that are targeted by the gRNA is 10 to 10,000.

7. The isolated cells according to claim 6, wherein the gRNA targets transposable elements (TEs).

8. The isolated cells according to claim 6, wherein the single-base editor is an adenine base editor comprising: a nickase or dead editor for A-to-G editing; and an adenosine deaminase.

9. The isolated cells according to claim 6, wherein the single-base editor is a cytosine base editor comprising: a nickase or dead editor for C-to-T editing; and a cytidine deaminase.

10. The isolated cells according to claim 6, wherein the gRNA comprises at least one sequence selected from the group consisting of SEQ ID NOs: 1 to 3716.

11. A method for measuring or estimating elapsed time in isolated cells, comprising steps of:
(a) transducing the composition of claim 1 into the isolated cells of claim 6 and then culturing the cells;
(b) measuring a copy number frequency of a sequence edited by the composition, that is, an A-to-G conversion frequency (CFᵢ) at an i^{th} target site;
(c) measuring a copy number frequency of an intact sequence in a total copy number of the edited sequence, that is, a frequency of intact sequence (Fᵢ) at the i^{th} target site; and
(d) measuring a time elapsed from a given time point using the following equation:
$${F}_{i} = 1 - {CF}_{i} = {e}^{- {λ}_{i} ( t - {t}_{0} )} ( t \geq 0 , {t}_{0} \geq 0 )$$
wherein Fᵢ represents the frequency (fraction) of the copy number of an intact sequence at the i^{th} target site relative to the total copy number, analyzed at any time point, CFᵢ represents the copy number fraction of the edited sequence at the i^{th} target site, measured at any time point, λi is a positive constant that represents the rate of editing at the i^{th} target site per unit time, and to is the latent time taken for the composition transduced into the cells to be expressed.

12. The method according to claim 11, further comprising a step of calculating λi using the following equation:
$${F}_{i} = {e}^{- {λ}_{i} {t}^{*}} ( {t}^{*} \geq 0 )$$
wherein Fᵢ represents the frequency (fraction) of the copy number of an intact sequence at the i^{th} target site relative to the total copy number, analyzed at any time point, λi is a positive constant, and t* is a positive constant that represents a given time point.


SEQUENCE LISTING (5 sequences)

SEQ ID NO: 1 - DNA, 4158 bp, synthetic construct
  misc_feature 1..4158: note = Synthetic Sequence
  source 1..4158: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 2 - DNA, 14 bp, synthetic construct
  misc_feature 1..14: note = Synthetic Sequence
  source 1..14: mol_type = other DNA
tcgccggagctgag

SEQ ID NO: 3 - DNA, 14 bp, synthetic construct
  misc_feature 1..14: note = Synthetic Sequence
  source 1..14: mol_type = other DNA
tggccggagctgag

SEQ ID NO: 4 - DNA, 169 bp, synthetic construct
  misc_feature 1..169: note = Synthetic Sequence
  source 1..169: mol_type = other DNA
gaggtatcgccggagctgaggttttagagctagaaatagcaagttaaaataaggctagtccgttatcaacttgaaaaagtggcaccgagtcggtgctcagctccggcgatacctcagctccggcgcgcggttctatctagttacgcgttaaaccaactagaattttttt

SEQ ID NO: 5 - DNA, 76 bp, synthetic construct
  misc_feature 1..76: note = Synthetic Sequence
  source 1..76: mol_type = other DNA
gaggtatcgccggagctgagtggccggagctgagtggccggagctgagtggccggagctgagtggccggagctgag